(19) United States
(12) Patent Application Publication
(10) Pub. No.: US 2001/0036664 A1
(43) Pub. Date: Nov. 1, 2001

(54) L-leucyl-L-leucine methyl ester treatment of donor lypmhocyte infusions in bone marrow transplant patients

(76) Inventors: Robert Korngold, Cherry Hill, NJ (US); Neal Flomenberg, Cherry Hill, NJ (US); Michael Hsieh, Philadelphia, PA (US)

Correspondence Address: THOMAS JEFFERSON UNIVERSITY, INTELLECTUAL PROPERTY DIVISION, 1020 WALNUT STREET, SUITE 620, PHILADELPHIA, PA, 19107, US

(21) Appl. No.: 09/803,223

(22) Filed: Mar. 9, 2001

Related U.S. Application Data
(60) Provisional application No. 60/188,391, filed on Mar. 10, 2000.

(51) Int. Cl. C12N 5/08

(52) U.S. Cl. 435/372; 424/93.7

(57) ABSTRACT

The present invention relates to a method of inhibiting graft-versus-host disease in allogeneic hematopoietic stem cell transplant (HSCT) patients by using L-leucyl-L-leucine methyl ester (LLME) to eliminate selective cytotoxic T cells in donor lymphocyte infusions (DLI). LLME has been shown to inhibit GVHD in animal models by selectively inducing apoptosis in natural killer cells and cytotoxic T cells. The application of LLME to the human clinical HSCT situation, however, has been hampered by HSC toxicity when unseparated marrow is treated at the concentrations necessary to purge GVHD-inducing T cells prior to infusion. In the present invention, this problem is circumvented by the LLME ex vivo treatment of DLI administered following transplantation of T cell-depleted HSC. In this setting, the effects of LLME on HSC contained within the DLI are irrelevant for clinical outcome. In another embodiment, the risk of toxicity to the stem cell population is avoided by ex vivo LLME treatment of donor lymphocytes after separation of CD34⁺ stem cells and then co-administration of the LLME-treated donor CD34⁻ fraction and the untreated CD34⁺ stem cells.

### CROSS REFERENCE TO RELATED APPLICATIONS

This application claims priority under 35 U.S.C. § 119 based upon U.S. Provisional Application No. 60/188,391 filed Mar. 10, 2000.

### GOVERNMENT RIGHTS TO THE INVENTION

The invention was made with government support under grant R01-HL-555593 awarded by the National Heart, Lung, and Blood Institute of the National Institutes of Health. The government has certain rights to the invention.

### FIELD OF THE INVENTION

The present invention generally relates to the fields of medicine and immunology and to a method of using L-leucyl-L-leucine methyl ester (LLME) to selectively eliminate cytotoxic T-cells in donor lymphocyte infusions (DLI) simultaneously with, as part of, or following allogeneic hematopoietic stem cell transplantation (HSCT) and, more particularly, to inhibiting the development of graft-versus-host-disease in mammals.

### ABBREVIATIONS

“LLME” means “L-leucyl-L-leucine methyl ester.”

“HSCT” means “hematopoietic stem cell transplantation.”

“HSC” means “hematopoietic stem cell.”

“DLI” means “donor lymphocyte infusion.”

“BMT” means “bone marrow transplant.”

“ATBM” means “T cell-depleted bone marrow cells.”

“GVHD” means “graft-versus-host-disease.”

### BACKGROUND OF THE INVENTION

Allogeneic hematopoietic stem cell transplantation (HSCT) is a unique modality in cancer therapy. While, in some settings, this treatment may be the only currently curative approach and may produce very low relapse rates, its high early mortality often dampens enthusiasm for its use. The consequences of graft versus host disease (GVHD) prophylaxis and treatment and, in particular, the immunoincompetence and vulnerability to secondary opportunistic infections of allogeneic HSCT recipients are the major reasons for the higher mortality in these patients. The hallmark of this immunodeficiency is a prolonged CD4⁺ T cell cytopenia. Current approaches for conditioning patients and T cell-depleting HSCT can secure consistent engraftment without any GVHD, but clinical outcome is still unsatisfactory because of immunoincompetence and secondary infections. Since GVHD can be prevented without the need for ongoing immune suppression, if immunoincompetence, particularly CD4⁺ cytopenia, can be rapidly reversed, then the mortality of allogeneic HSCT will drop to levels more comparable to those of autologous HSCT, producing an improved therapeutic index.

Immunoincompetence after HSCT.

Immunodeficiency is a major problem after allogeneic bone marrow transplantation (BMT) whether pharmacoprophylaxis or T cell depletion is used for GVHD prophylaxis. (Blume, K. G., Leukemia 7:1078, 1993; Armitage, J. O., New England Journal of Medicine 330:827-838, 1994; Thomas, E. D., Perspectives in Biological Medicine 38:230-237, 1995). In particular, sustained depression of CD3⁺ and CD4⁺ counts are common in adults. (Brown, R. A., et al., Journal of Clinical Onclolgy 15:3067-3074, 1997; Pavletic, Z. S., et al., Journal of Clinical Oncology 15:1608-1616, 1997). CD8⁺ cells normalize earlier, often within 30-60 days after T cell depletion or unmodified BMT. (Pavletic, Z. S., et al., Journal of Clinical Oncology 15:1608-1616, 1997). With the use of more aggressive approaches for GVHD prevention in the haploidentical setting, immunodeficiency becomes more severe and long lasting. Secondary infections, Epstein-Barr Virus-lymphoproliferative disorder (EBV-LPD), and CMV viremia all occur with increased frequency in patients with low CD4⁺ counts. (Brown, R. A., et al., Journal of Clinical Oncology 15:3067-3074, 1997; Pavletic, Z. S., et al., Journal of Clinical Oncology 15:1608-1616, 1997). This scenario is thought to be similar to the risk of infection associated with low CD4⁺ counts in HIV⁺ patients. In the HIV population, a lower CD4⁺ count is associated with increased risk of infection and is an independent predictor of the inability of HIV to respond to any anti-viral drug therapy. (Haase, A. T., Annual Review of Immunology 17:625-656, 1999). Even surgical infectious complications are inversely correlated to the CD4⁺ count. (Savioz, D., et al., European Journal of Surgery 164:483-487, 1998). These data lead to the hypothesis that more rapid recovery of CD4 cell numbers is associated with lower incidences of life threatening infection and better survival post transplant.

DLI for Immune Reconstitution.

Refractory viral diseases in the immunocompromised host have been treated by the infusion of unmanipulated donor lymphocytes. Donor lymphocyte infusion (DLI) therapy after transplant has been used for a variety of infections including persistent adenovirus (Hromas, R., et al., Blood 84:1689-1690, 1994), CMV (Witt, V., et al., Bone Marrow Transplantation 22:289-292, 1998), EBV-LPD (Papadopoulos, E. B., et al., New England Journal of Medicine 330:1185-1191, 1994), and hepatitis (Shouval, D. & Ilan Y., Journal of Hepatology 23(1):98-101, 1995). Several groups have documented the utility of both virus specific and unmanipulated donor lymphocytes to restore T-cell numbers, (Small, T. N., et al., Blood 93:467-480, 1999), and to successfully treat infection or relapse (Dazzi, F. & Goldman, J. M., Annual Review of Medicine 49:329-340, 1998; Pati, A. R., et al., Bone Marrow Transplantation 15:979-81, 1995). One major complication of unmanipulated DLI therapy is GVHD. (Dazzi, F. & Goldman, J. M., Annual Review of Medicine 49:329-340, 1998). The development of GVHD following DLI is correlated with (a) higher doses of T cells, (b) greater degrees of immunogenetic disparity, and (c) shorter time from transplant to DLI. In HLA matched siblings, the threshold dose above which GVHD is likely is approximately 10⁵ CD3⁺ cells/kg of recipient body weight if given at the time of transplant. In contrast, doses of 10⁷ CD3+ cells/kg administered more than 9 months after BMT generally do not produce GVHD. In recipients of mismatched marrow, however, DLI doses of 2.4×10⁵/kg administered 1-2 months after BMT produced a 56% incidence of severe acute GVHD despite continuation of immune suppression. (Dazzi, F. & Goldman, J. M., Annual Review of Medicine 49:329-340, 1998). This highlights the need to vary T cell dosing based on time after BMT and immunogenetic disparity.

LLME.

L-leucyl-L-leucine methyl ester (LLME) is an agent that selectively eliminates those T cells containing cytotoxic effector granules. LLME is taken up by cells through saturable facilitated transport. (Thiele, D. L. & Lipsky, P. E., Journal of Experimental Medicine 172(1): 183-94, 1990). Once intracellular, dipeptidyl peptidase I (DPP_{I}), expressed primarily by cytotoxic granule-containing leukocytes, converts LLME to pro-apoptotic (Leu-Leu)ₙ-OMe metabolites, killing the cells. Thus, LLME induces programmed cell death of most natural killer (NK) cells, monocytes, granulocytes, and the majority of CD8⁺ T cells but only a small fraction of CD4⁺ T cells. (Thiele, D. L. & Lipsky, P. E., Proceedings of the National Academy of Sciences of the United States of Ameica 87(1):83-7, 1990).

LLME has demonstrated salutary effects in preventing GVHD in animal models. Ex vivo treatment of bone marrow grafts with LLME can completely prevent GVHD in multiple murine models (Thiele, D. L., et al., Journal of Immunology 138(1):51-7, 1987; Blazar, B. R., et al., Blood 75(3):798-805, 1990) such that overt GVHD does not develop, and histopathological evidence is restricted to mild to moderate cholangitis (Willams, F. H. & Thiele, D. L., Hepatology 19(4):980-8, 1994) and transient skin infiltrates with mild residual dermal sclerosis. In canine studies, similar desirable effects of LLME on T cells and GVHD incidence were noted, but, unlike the mouse, evidence of stem cell toxicity was also seen. (Raff, R. F., et al., Transplantation 46(5):655-60, 1988; Kiem, H. P., et al., Blood 88(5):1896-7, 1996). LLME depletes human NK/LAK cells, monocytes, granulocytes, and selected T cell subpopulations, but also reduces human colony forming units (CFU). (Pecora, A. L., et al., Journal of Immunology 136(3):1038-48, 1986). The application of LLME to the human clinical HSCT situation has been severely limited by toxicity to HSC when unseparated marrow was treated prior to infusion. (Rosenfeld, C. S., et al., Transplantation 60(7):678-83, 1995). There is consequently a need for circumventing this toxicity problem to enable the use of LLME in patients requiring bone marrow transplants and DLI.

The present invention relates to a method of inhibiting GVHD and other toxic effects of T cell infusions in bone marrow transplant patients by ex vivo LLME treatment of DLI administered at the time of or following T cell depleted bone marrow transplantation.

### SUMMARY OF THE INVENTION

It is an object of the present invention to inhibit GVHD in a mammal requiring DLI. The donor lymphocytes to be infused are contacted with an aqueous solution containing a therapeutically effective amount of LLME ex vivo, selective cytotoxic T cells are eliminated, and the donor lymphocytes then are infused into the mammal, thereby inhibiting GVHD. In one embodiment, DLI is required following allogeneic T cell depleted HSCT. In another embodiment, infusion of the donor lymphocytes into the mammal occurs on the same day as administration of allogeneic T cell-depleted HSCT. In another embodiment, infusion of the donor lymphocytes into the mammal occurs after HSC engraftment. In a further embodiment of the present invention, the mammal is a human.

Another object of the present invention is to inhibit GVHD in a mammal requiring transplant of CD34⁺ stem cells. The HSC to be infused are separated into CD34⁻ and CD34⁺ fractions. The CD34⁻ HSC fraction is contacted with an aqueous solution containing a therapeutically effective amount of LLME ex vivo, selective cytotoxic T cells in the CD34⁻ fraction are eliminated, and a therapeutically effective amount of the LLME-treated CD34⁻ HSC fraction then is co-administered with the untreated CD34⁺ HSC fraction to the mammal, thereby inhibiting GVHD.

### BRIEF DESCRIPTION OF THE FIGURES

FIG. 1. (B6×DBA/2)F₁ (H2^{b/d} haplotype) mice are lethally irradiated (13 Gy split-dose) and injected with 2×10⁶ C57B1/6 (H2^{b}) T cell-depleted bone marrow cells (ATBM) on day—14. On day 0, the mice are injected intravenously with a DLI of either 2×10⁷, 4×10⁷ or 1.5×10⁸ splenocytes from presensitized C57B1/6 mice. The administered splenocytes are either mock-treated (washed, but not treated) or treated ex vivo with LLME.

FIG. 2. (B6×DBA/2)F₁ (H2^{b/d} haplotype) mice are lethally irradiated (13 Gy split-dose) and injected with 2×10⁶ B6 ATBM on day—14. On day—1, half of the recipients are challenged intraperitoneally with 1×10⁶ MMD2-8 myeloid leukemia cells. On day 0, mice are injected intravenously with a DLI of 4×10⁷ splenocytes from presensitized C57B1/6 mice. The administered splenocytes are either mock-treated (washed, but not treated) or treated ex vivo with LLME.

### DETAILED DESCRIPTION

Clinical application of LLME previously has been hampered by stem cell toxicity at the concentrations (500 μM) necessary to purge GVHD-inducing T cells. The present invention circumvents this problem by ex vivo LLME treatment of DLI administered following transplantation of T cell depleted bone marrow. In this setting, effects of LLME on the stem cells contained within DLI are irrelevant for clinical outcome since donor HSC engraftment already has occurred in the recipient.

Methods

A haploidentical model featuring C57B 1/6 (B6) donor mice (H2^{b}), (B6×DBA/2)F₁ (B6D2) recipient mice (H2^{b/d}), and the MMD2-8 myeloid leukemia line of DBA/2 origin (H2^{d}) is employed. Donor mice are presensitized against the recipient's splenocytes to increase GVHD risk.

In order to assess the ability of LLME-treated DLI to cause GVHD, B6D2 recipient mice are administered a split-dose of lethal irradiation (13 Gy split-dose) and reconstituted with 2×10⁶ C57B1/6 (H2^{b}) T cell-depleted bone marrow cells (ATBM). Fourteen (14) days later the recipient mice are injected intravenously with DLI of either 2×10⁷, 4×10⁷, or 1.5×10⁸ splenocytes from C57B1/6 mice that had been presensitized with (B6×DBA/2)F₁ cells. The administered splenocytes are either untreated (washed, but not treated) or treated ex vivo with LLME (incubation for 15 minutes at 2.5 million cells/ml of LLME375 μM solution).

The ability of LLME-treated DLI to mediate graft-versus-tumor responses is assessed using MMD2-8 leukemia-challenged mice. As above, B6D2 recipient mice are irradiated (13 Gy split-dose) and injected with 2×10⁶ C57B1/6 (H2^{b}) ATBM. After 13 days, half of the recipients are challenged intraperitoneally with 1×10⁶ MMD2-8 myeloid leukemia cells (H2^{d}) derived from the DBA/2 strain of mice. The following day, recipient mice are injected intravenously with 2×10⁷ splenocytes from C57B1/6 mice that had been presensitized with (B6×DBA/2)F₁ cells. The administered splenocytes are either untreated (washed, but not treated) or treated ex vivo with LLME (incubation for 15 minutes at 2.5 million cells/ml of LLME375 μM solution).

Results

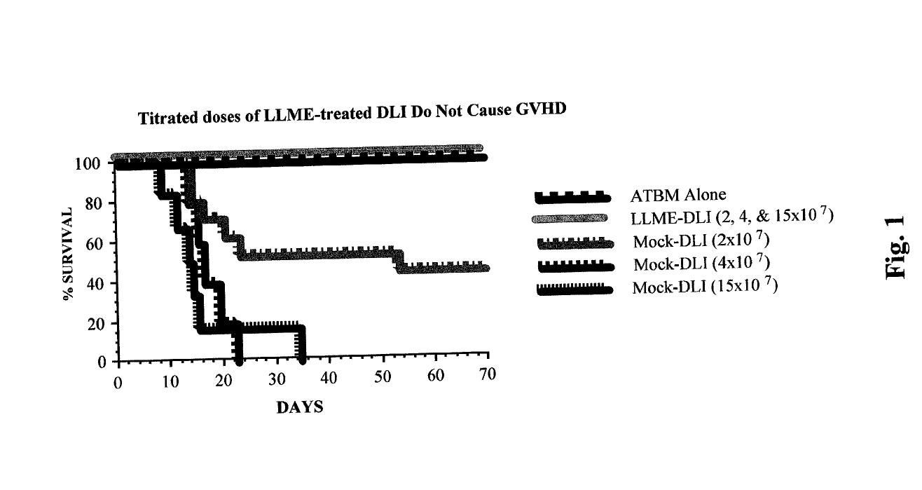

The ability of LLME-treated DLI to cause GVHD is assessed with graded doses of B6 donor splenocytes B6D2 recipients are irradiated to destroy lymphocytes and then reconstituted with B6 ATBM. Fourteen days later, the recipients receive DLI (2×10⁷, 4×10⁷, or 1.5×10⁸ cells). Mice receiving any dose of LLME-treated DLI experience increased survival (FIG. 1), with intact proliferative responses to LPS, enhanced donor chimerism, and neither cachexia nor lymphoid hypoplasia. In contrast, mice receiving mock-treated DLI at the mid to higher doses [4×10⁷ (n=5) and 1.5×10⁸ (n=6) cells] experience high mortality and expression of the other typical parameters of disease. At the lower dose of 2×10⁷ mock-treated splenocytes (n=11), 45% of the mice survive but exhibit symptoms of GVHD.

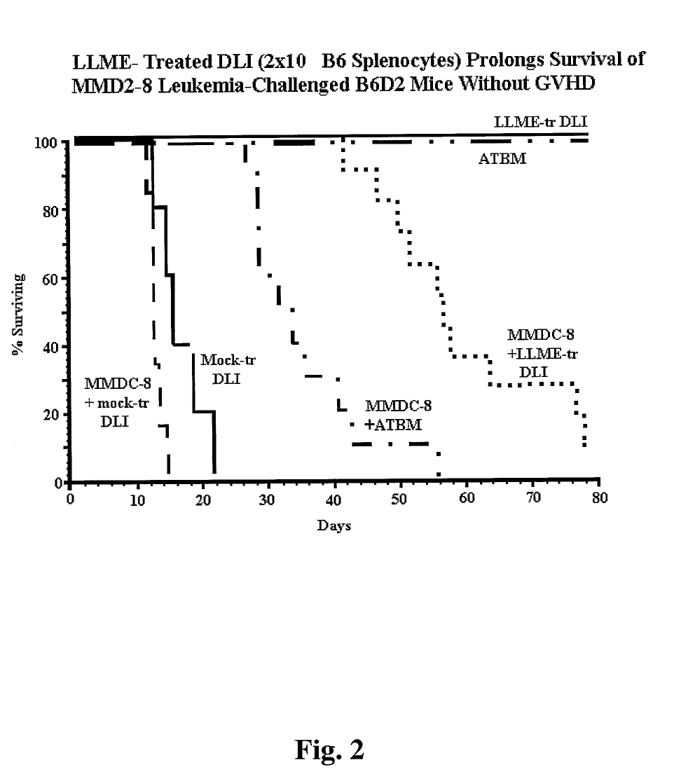

The ability of LLME-treated DLI to mediate graft-versus-tumor responses is assessed using MMD2-8 leukemia-challenged mice. Mice receiving LLME-treated DLI splenocytes (n=11), but no tumor challenge, experience significantly increased survival (p<0.05) compared to tumor-challenged mice without DLI (n=10), while mice receiving mock-treated DLI plus tumor challenge (n=6) die rapidly from GVHD. (FIG. 2). Mice treated with mock-treated DLI alone have a median survival time of 12 days with 0% survival (primarily due to GVHD) compared to the LLME-DLI group (n=11), which had a median survival of 56 days and 10% survivors at day 79 post-transplant. Recipients of ATBM challenged with tumor alone have a median survival of 34 days with 0% survival (due to leukemia burden). Disease is confirmed through body weight changes, histological analysis, and flow cytometry (lymphoid hypoplasia).

These results demonstrate the potential ability of LLME treated T cells to augment immune responses to antigens, including malignant cells, in transplanted animals without induction of GVHD.

Discussion

The subject of the present invention is preferably an animal, including but not limited to, animals such as pigs, monkeys, etc., and is preferably a mammal, and most preferably human. The efficacy of LLME in preventing GVHD and its sparing of CD4⁺ T cells implies that infusions of LLME-treated T cells will rapidly reverse the CD4⁺ cytopenia seen after transplant. This can best be achieved by administering DLI treated with a therapeutically effective amount of LLME approximately 30 days after HSCT, when engraftment already has occurred. Alternatively, LLME-treated DLI can be added at time of transplant of stem cells, whereby only donor lymphocytes are treated after separation of CD34⁺ stem cells. CD34⁺ stem cells thus will be allowed to reconstitute the hematopoietic/lymphoid compartment of the recipient unhindered by any potential risk of toxicity related to ex vivo LLME treatment. It would be anticipated that the CD34⁺ fraction will rapidly reconstitute CD8⁺ T cells, while the LLME treated CD34⁻ fraction will similarly reconstitute the CD4⁺ T cell subset. HSC can be separated into CD34⁺ and CD34⁻ fractions using current CD34 column separation technology. The CD34⁺ fraction is then administered untreated, either by itself, or along with an appropriate number of LLME-treated T cells from the CD34⁻ fraction. For delayed infusions, LLME-treated donor CD34⁻ fractions can be cryopreserved for later time points or fresh cells can be collected at the time of delayed administration. For ex vivo LLME treatment of DLI, a biologically effective level of LLME varies from circumstance to circumstance but generally lies between about 1 micromolar and about 250 micromolar. The addition of LLME-treated T cells will provide improved donor CD4⁺ T cell counts with GVHD control, thereby providing a lower risk of opportunistic infections and a better platform for graft-versus-tumor manipulations.

Currently, bone marrow transplantation is used as a major mode of therapy in treating aplastic anemia, acute myelogenous leukemia, and a variety of immunodeficiency states. As mentioned above, a major complication of this therapy is GVHD. Current regimens for the prevention and treatment of GVHD consist of depleting T-lymphocytes from the donor marrow prior to transplantation and giving the recipient immunosuppressive drugs such as cyclophosphamide and methotrexate, both before and after transplantation. Both regimes result in immunodeficiency and vulnerability to secondary infections, the frequency of which is associated with low CD4⁺ counts. Treatment of refractory viral diseases in the immunocompromised host involves unmanipulated DLI, one major complication of which is GVHD. Thus, by virtue of its ability to induce programmed cell death of NK cells and cytotoxic T cells, but only a small fraction of CD4⁺ T cells, ex vivo LLME treatment of DLI administered following BMT will be efficacious in diminishing this complication. An effective level of LLME in the present invention for ex vivo induction of apoptosis of NK cells and selective cytotoxic T cells is between about 10 micromolar and about 250 micromolar. This prediction of effective GVHD prevention is supported by experiments described supra.

A second problem in bone marrow transplantation is the risk of secondary infection. Clonal populations of T cells have been administered after transplant in the hopes of treating or preventing infections such as CMV. Such clones usually have disappeared from the blood stream after several weeks. It has been hypothesized that this disappearance reflects lack of T-cell help, either specific or non-specific, to facilitate expansion or persistence of the clones. The improved immunologic environment that ex vivo LLME treatment of DLI provides will enhance the effectiveness of these approaches for the prevention or treatment of, among other things, infectious complications, post transplant lymphoproliferative disorder, and relapse of the underlying malignancy for which the transplant was performed.

Current regimens for the prevention and treatment of post transplant infections complications and residual malignancy include vaccine therapy. The improved immunologic environment that LLME ex vivo treatment of DLI provides will provide an enhanced platform for all immunization strategies that might be employed.

Other clinical uses for the present invention are other situations in which allogeneic marrow/stem cell transplantation is utilized. For example in solid organ transplants in general (kidney, heart, liver, pancreas, skin, etc.), it is widely accepted that cytotoxic T cells are likely to be the cell type responsible for graft rejection. (Mayer, et al., J. Immunol. V 134:258). Allogeneic BMT has been utilized in this clinical setting to promote immunologic tolerance. Thus, it is contemplated that the ex vivo LLME treatment of DLI will benefit in preventing allograft rejection by securing rapid engraftment and rapid immune reconstitution after allogeneic HSCT.

It is also contemplated that the method of the present invention will be of benefit in other spontaneously occurring disease states. A variety of diseases have been classified as “autoimmune diseases” because of the widely accepted belief that they are caused by disorders in the immune system that cause immunologic damage to “self”. Thus, in a variety of diseases, including, but not limited to, primary biliary cirrhosis, systemic lupus erythematosus, rheumatoid arthritis, multiple sclerosis, autoimmune hemolytic anemia, etc., various forms of immunologic damage to selected organs occur. In some of these diseases, such as primary biliary cirrhosis, the histologic abnormalities that occur (in this case in the liver) closely resemble those that occur in GVHD or in rejection of a transplanted liver (Fennel, Pathol. Annu. V 16:289 (1981). It is reasonable that benefit from allogeneic HSCT should also occur in such disease states. Consequently, it is contemplated that the ex vivo treatment of DLI with LLME will enhance such treatment.

The method of the present invention may also be used to enhance the effectiveness of allogeneic HSCT treatment for hemoglobinopathies, thalessemia, aplastic anemia, and other types of bone marrow dysfunction. To date, allogeneic HSCT has been limited to only the highest risk of these applications because these have been the patients for whom the potential benefits of the procedure balance the potential risks. The ability to secure rapid engraftment and rapid immune reconstitution after allogeneic HSCT, with an improved overall safety, would allow broader application of this treatment approach to these additional therapeutic targets. Thus, the effectiveness of HSCT treatment will be enhanced by ex vivo LLME treatment of DLI administered following transplantation of T cell depleted bone marrow.

We claim:

1. A method of inhibiting GVHD in a mammal requiring DLI, the method comprising
a) contacting the donor lymphocytes to be infused with an aqueous solution containing a therapeutically effective amount of LLME ex vivo;
b) eliminating selective cytotoxic T-cells;
c) infusing said donor lymphocytes into said mammal; and
d) inhibiting GVHD.

2. The method of claim 1, wherein said mammal requires DLI following allogeneic T cell-depleted HSC.

3. The method of claim 1, wherein said infusing of said donor lymphocytes into said mammal occurs after donor HSC engraftment.

4. The method of claim 1, wherein said mammal is a human.

5. A method of inhibiting GVHD in a mammal requiring transplant of CD34⁺ stem cells, said method comprising
a) separating the HSC to be infused into CD34⁺ and CD34⁻ fractions;
b) contacting said CD34⁻ HSC fraction with an aqueous solution containing a therapeutically effective amount of LLME ex vivo;
c) eliminating selective cytotoxic T-cells in the CD34⁻ HSC fraction
d) co-administering a therapeutically effective amount of said LLME-treated CD34⁻ HSC fraction with said CD34⁺ HSC fraction; and
e) inhibiting GVHD.
